(19) United States
(12) Patent Application Publication
(10) Pub. No.: US 2025/0345487 A1
(43) Pub. Date: Nov. 13, 2025

(54) COMPOSITE HYDROGEL AND USES THEREOF

(71) Applicant: National Taiwan University, Taipei (TW)

(72) Inventors: Jia-Shing YU, Taipei (TW); Ta-Ching CHEN, Taipei (TW); Yi-Chen LIU, Taipei (TW)

(21) Appl. No.: 18/957,429

(22) Filed: Nov. 22, 2024

(30) Foreign Application Priority Data
May 13, 2024 (TW) 113117582

(51) Int. Cl. A61L 27/38; A61L 27/02; A61L 27/18; A61L 27/52; C12N 5/0775

(52) U.S. Cl. CPC A61L 27/3834; A61L 27/025; A61L 27/18; A61L 27/52; C12N 5/0662; A61L 2400/12; C12N 2500/90; C12N 2533/30; C12N 2533/54

(57) ABSTRACT

A composite hydrogel includes gelatin, cinnamic acid, polydopamine nanoparticles, and curcumin is provided. The gelatin is chemically cross-linked with the cinnamic acid to form cinnamic acid-modified gelatin (GelCA), and the curcumin is non-covalently linked to the polydopamine nanoparticles to form curcumin-loaded polydopamine nanoparticles (Cur@PDA). The GelCA undergoes a photo-crosslinking process and then binds with the Cur@PDA to form the composite hydrogel. A method for treating retinal tissue damage includes administering to a subject in need thereof an effective amount of the composite hydrogel.

### CROSS-REFERENCES TO RELATED APPLICATIONS

This application claims the benefit of priority to Taiwanese Patent Application No. 113117582 filed on May 13, 2024, which is hereby incorporated by reference in its entirety.

### BACKGROUND OF THE INVENTION

### Field of the Invention

The present invention relates to a composite hydrogel and a method for treating retinal tissue damage, particularly to a composite hydrogel composed of a gelatin matrix modified with cinnamic acid and curcumin-loaded polydopamine nanoparticles, formed through a photo-crosslinking reaction, and a method for treating retinal tissue damage by applying the composite hydrogel.

### Descriptions of the Related Art

Hydrogels are polymer materials widely used in the medical field, known for their high water absorption, biocompatibility, and adjustability. The common constituents of hydrogels include polyethylene glycol (PEG), polyacrylic acid (PAA), polyvinyl alcohol (PVA), and polylactic acid-polyethylene glycol (PLA-PEG). It's worth noting that hydrogels derived from natural materials such as gelatin, sodium alginate, and collagen exhibit superior biocompatibility and biodegradability. These materials provide soft tissue support without causing adverse reactions to surrounding tissues, thereby aiding in tissue repair and drug delivery. The high water retention and plasticity of hydrogels make them extensively applicable in wound dressings, drug delivery, tissue engineering, and other fields.

The retinal tissue is a crucial component of the visual system, responsible for converting light signals into visual perceptions that the brain can understand. However, this delicate tissue is susceptible to oxidative damage, particularly damage caused by reactive oxygen species (ROS). Retinal oxidative stress is a significant pathological process in eye diseases. When the retina is subjected to oxidative stress, excessive free radicals and oxidative substances are produced, leading to damage in cell structure and function, which can trigger various retinal diseases, including macular degeneration, diabetic retinopathy, retinal artery occlusion, and glaucoma. Therefore, developing treatments that can mitigate or prevent retinal oxidative stress damage is of paramount importance.

The focus of research on treating retinal diseases related to damage is on developing therapeutic drugs that are effective, locally concentrated, and long-acting. Currently, the most common methods for treating retinal diseases include directly administering small molecule drugs to the affected area or delivering drugs to the vitreous through the bloodstream. The primary treatment options currently remain the antioxidants, anti-inflammatory drugs, and growth factors from conventional therapies. Antioxidants help neutralize free radicals and reduce oxidative stress. Anti-inflammatory drugs can suppress inflammatory responses and reduce tissue damage. Growth factors promote the repair and regeneration of damaged tissues. However, these treatment methods may face challenges such as low bioavailability, absorption issues, and limitations in drug solubility, which can reduce therapeutic efficacy and increase the risk of further damage to the affected area.

In view of the above, the present invention provides a composite hydrogel to improve the low bioavailability and the need for repeated injections associated with drugs used in the treatment of retinal diseases in the prior art. These issues can lead to problems such as tissue damage and diminished therapeutic efficacy due to repeated injections.

### SUMMARY OF THE INVENTION

An objective of the present invention is to provide a novel composite hydrogel, which undergoes unique group modifications. The composite hydrogel includes gelatin modified with cinnamic acid molecules, where the carboxyl groups in the cinnamic acid molecule structure chemically crosslink with the lysine residues in the gelatin, as well as curcumin-loaded polydopamine nanoparticles. The composite hydrogel of the present invention is particularly formed by a photo-crosslinking reaction under ultraviolet light irradiation. The phenyl groups of cinnamic acid in the hydrogel material enhance the affinity of the hydrogel for lipophilic molecules, and the alkene functional groups of cinnamic acid can undergo photo-dimerization reactions via a photoinitiator, resulting in a gel material with superior viscoelastic properties. This makes it particularly suitable as an injectable carrier for drug delivery systems. Furthermore, the lipophilic curcumin is adsorbed onto the polydopamine nanoparticles through x-x stacking interactions and hydrogen bonding, allowing for effective distribution of curcumin within the composite hydrogel. This increases the bioavailability and absorption of curcumin in tissues within the human body. Thus, the present invention provides a composite hydrogel with excellent bioavailability, injectability, tissue adhesion, and antioxidant properties, which can enhance the therapeutic efficacy of curcumin in retinal tissue.

To achieve the above object, the present invention provides a composite hydrogel. The composite hydrogel includes a gelatin, a cinnamic acid, a plurality of polydopamine nanoparticles, and a curcumin. The gelatin is chemically cross-linked with the cinnamic acid to form a cinnamic acid-modified gelatin (GelCA). The curcumin is linked by a non-covalent binding method to the polydopamine nanoparticles to form a plurality of curcumin-loaded polydopamine nanoparticles (Cur@PDA). The cinnamic acid-modified gelatin undergoes a photo-crosslinking reaction and then binds with the curcumin-loaded polydopamine nanoparticles to form the composite hydrogel.

In one embodiment, the gelatin is chemically cross-linked with the cinnamic acid by a chemical crosslinking agent forming an amide bond between the carboxyl group of the cinnamic acid and an amino group of the gelatin to form a covalent bond.

In one embodiment, the chemical crosslinking agent is 1-ethyl-3-(3-dimethylaminopropyl) carbodiimide (EDC).

In one embodiment, the composite hydrogel further comprises a photoinitiator and a deionized water, and the photoinitiator is lithium phenyl-2,4,6-trimethylbenzoylphosphinate (LAP).

In one embodiment, the photo-crosslinking reaction is initiated by an ultraviolet irradiation of the photoinitiator and generates a photo-dimerization reaction at the alkene functional group in the cinnamic acid-modified gelatin.

In one embodiment, the polydopamine nanoparticles are formed through the self-polymerization of a dopamine.

In one embodiment, based on the total weight of the composite hydrogel, the weight percentage of the curcumin-loaded polydopamine nanoparticles is less than 10 wt %.

In one embodiment, the non-covalent binding method includes T-T stacking and hydrogen bonding.

In one embodiment, the composite hydrogel is injectable.

The present invention further provides a method for treating retinal tissue damage, comprising administering to a subject in need thereof an effective amount of the composite hydrogel. The composite hydrogel is primarily composed of gelatin modified with cinnamic acid, serving as the hydrogel matrix, and incorporates polydopamine nanoparticles loaded with curcumin. The modified composite hydrogel, formed through a photo-crosslinking reaction, has the capability to adsorb drugs with similar stacking properties. As a result, the hydrogel demonstrates superior bioavailability, biocompatibility, excellent tissue adhesion, and durability. It can effectively scavenge reactive oxygen species in retinal tissue, thereby enhancing its antioxidant capacity.

To achieve the aforementioned objectives, the present invention provides a composite hydrogel and a method for applying the composite hydrogel to treat retinal tissue damage.

After referring to the drawings and the embodiments as described in the following, those the ordinary skilled in this art can understand other objectives of the present invention, as well as the technical means and embodiments of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of the preparation process of the composite hydrogel of the present invention;

FIG. 2 shows images of the polydopamine nanoparticles (PDA) and the curcumin-loaded polydopamine nanoparticles (Cur@PDA) of the present invention observed using a scanning electron microscopy (SEM) and a transmission electron microscopy (TEM);

FIG. 3 is a schematic diagram showing the changes in the rheological properties of the cinnamic acid-modified gelatin (GelCA) of the present invention;

FIG. 4 is a schematic diagram showing the changes in compressive strength of the cinnamic acid-modified gelatin of the present invention;

FIG. 5 is a schematic diagram showing the swelling test of the cinnamic acid-modified gelatin of the present invention;

FIG. 6 is a schematic diagram showing the changes in the rheological properties of the composite hydrogel of the present invention;

FIG. 7 is a schematic diagram showing the changes in compressive strength of the composite hydrogel of the present invention;

FIG. 8 is a schematic diagram showing the changes in the long-term dissolution test of the composite hydrogel of the present invention;

FIG. 9 is a schematic diagram showing the changes in the short-term degradation test of the composite hydrogel of the present invention;

FIG. 10 is a schematic diagram showing the DPPH⋅ free radical scavenging activity of the polydopamine nanoparticles and the curcumin-loaded polydopamine nanoparticles of the present invention;

FIG. 11 is a schematic diagram showing the superoxide anions (O₂^{•—}) scavenging activity of the polydopamine nanoparticles and the curcumin-loaded polydopamine nanoparticles of the present invention;

FIG. 12 is a schematic diagram showing the changes in cell viability of RGC-5 cells treated with the composite hydrogel of the present invention;

FIG. 13 is a schematic diagram showing the changes in cell viability of R661W cells treated with the composite hydrogel of the present invention;

FIG. 14 shows tissue staining images of the composite hydrogel of the present invention and the control group 7 days after injection into retinal tissue;

FIG. 15 shows tissue staining images of the composite hydrogel of the present invention and the control group 14 days after injection into retinal tissue;

FIG. 16 is a schematic diagram showing the quantitative analysis of the relative fluorescence intensity using dihydroethidium (DHE) staining in retinal tissue treated with the composite hydrogel of the present invention; and

FIG. 17 is a schematic diagram showing the quantitative analysis of Brn3C-positive cell counts using immunofluorescence staining in retinal tissue treated with the composite hydrogel of the present invention.

### DETAILED DESCRIPTION OF THE PREFERRED EMBODIMENT

Reference will now be made in detail to the embodiments of the present invention, examples of which are illustrated in the accompanying drawings, and are not intended to limit the present invention, applications, or implementations described in these embodiments. Wherever possible, the same reference numbers are used in the drawings and the description to refer to the same or like parts. It should be noticed that, in the following embodiments and the attached drawings, elements unrelated to the present invention are omitted from the depiction; and dimensional relationships among individual elements in the drawings are provided only for ease of understanding, but not to limit the actual scale.

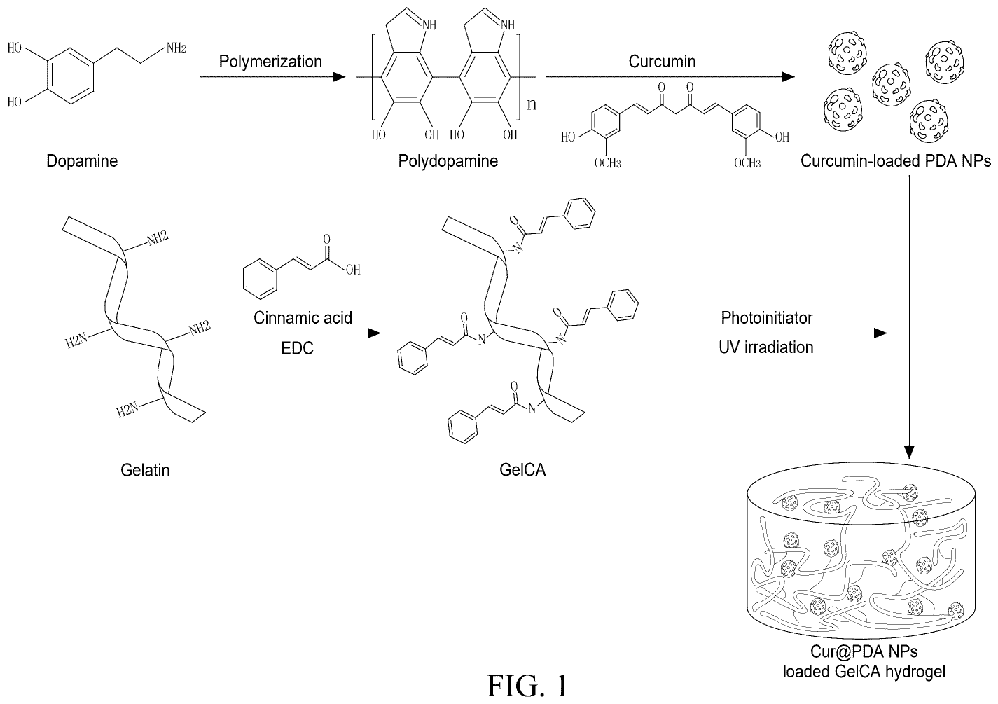

Please refer to FIG. 1, which shows a schematic diagram of the preparation process for the composite hydrogel of the present invention. The composite hydrogel of the present invention includes the preparation steps of forming the polydopamine nanoparticles through the polymerization of dopamine, and then reacting with curcumin, the preparation steps of obtaining cinnamic acid-modified gelatin through the chemical crosslinking reaction of gelatin and cinnamic acid, and the further step of forming a modified gelatin by photo-crosslinking the previously obtained cinnamic acid-modified gelatin, which is then combined with the curcumin-loaded polydopamine nanoparticles to form the composite hydrogel. The detailed synthesis steps are described as follows.

### Example 1

### [Preparation Method]

### [Preparation of Cinnamic Acid-Modified Gelatin (GelCA) Macromonomer]

The preparation of modified gelatin monomers involves using gelatin (type B), cinnamic acid, and the chemical crosslinking agent 1-ethyl-(3-dimethylaminopropyl) carbodiimide (EDC). This modified gelatin can serve as the hydrogel matrix for the composite hydrogel of the present invention. First, 0.0035 mol of trans-cinnamic acid was dissolved in 20 mL of 2 N sodium hydroxide (NaOH) solution, and the pH of the solution was precisely adjusted to 8. Then, 0.043 mol of EDC was dissolved in 60 mL of PBS solution at 4° C. to ensure EDC was evenly dispersed. The two solutions are then mixed and stirred at 4° C. for 30 minutes. In the next step, 5 g of gelatin (type B) was added to 50 mL of PBS solution and heated to 60° C. to ensure the gelatin was evenly dispersed in the solution. After cooling to room temperature, the previously prepared mixture of the two solutions at 4° C. was added to the gelatin solution and stirred at room temperature for 24 hours. Upon completion, 6-8 kDa dialysis bags were used to separate the reactant chemicals from the GelCA macromonomer. Finally, the GelCA macromonomer was lyophilized and securely stored in a drying cabinet. It should be noted that the molar ratio of gelatin, cinnamic acid, and 1-ethyl-(3-dimethylaminopropyl) carbodiimide (EDC) used in the present invention is 1:9:90.

Additionally, the presence of GelCA was confirmed by nuclear magnetic resonance (NMR) spectroscopy. NMR analysis revealed that GelCA was formed through the amide bond between the carboxyl group of cinnamic acid and the amino group (lysine) of gelatin, resulting in the covalent attachment of natural cinnamic acid to gelatin, forming GelCA. On the other hand, the degree of substitution of GelCA was calculated as 91% using the 2,4,6-trinitrobenzene sulfonic acid (TNBS) assay and the BCA protein assay (not shown in the figures), indicating that GelCA was a macromonomer with a high degree of substitution.

### [Preparation of Curcumin-Loaded Polydopamine Nanoparticles (Cur@PDA)]

First, 1 g of dopamine hydrochloride was dissolved in 40 mL of deionized water. Then, this solution was mixed with 4 mL of ammonium hydroxide, 160 mL of water, and 80 mL of ethanol. The mixture was stirred at room temperature for 24 hours to self-polymerize dopamine. Afterward, the solution was centrifuged and lyophilized to separate and collect the polydopamine nanoparticles. It should be noted that the small molecule drug curcumin could bind to the phenolic groups on polydopamine nanoparticles through non-covalent interactions such as hydrogen bonding and x-x stacking. Next, to prepare the synthesis of the curcumin-loaded polydopamine nanoparticles, 10 mg of curcumin was thoroughly dispersed in 2 mL of methanol. Then, 10 mg of polydopamine nanoparticles was prepared in 8 mL of deionized water. The curcumin solution was gradually added to the polydopamine nanoparticle mixture, followed by an additional 6 mL of methanol. After mixing and reacting for 24 hours, the resulting curcumin-loaded polydopamine nanoparticles (Cur@PDA) were collectd by centrifugation, followed by appropriate washing steps, and finally lyophilized for use.

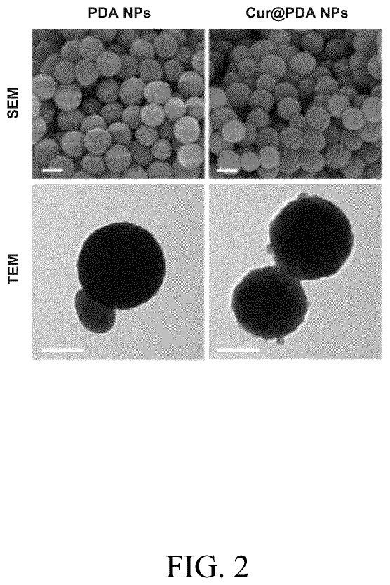

Therefore, the polydopamine nanoparticles and curcumin-loaded polydopamine nanoparticles were observed using scanning electron microscopy (SEM) and transmission electron microscopy (TEM), respectively, as shown in FIG. 2. SEM observations showed that both the polydopamine nanoparticles and the curcumin-loaded polydopamine nanoparticles had uniform particle sizes. However, the surface of the curcumin-loaded polydopamine nanoparticles became rougher, and irregularly shaped curcumin particles were observed. Furthermore, TEM observations showed that the polydopamine nanoparticles exhibited uniform electron transmittance and appeared as uniformly black spherical shapes. In contrast, for curcumin-loaded polydopamine nanoparticles, a discernible presence of curcumin on the exterior of the polydopamine nanoparticles was noted due to the difference in electron transmittance between curcumin and polydopamine.

### [Preparation of the Composite Hydrogel of the Present Invention]

First, a specified amount of curcumin-loaded polydopamine nanoparticles were dissolved in 900 μL of deionized water. Then, an ultrasonic homogenizer (Sonic Ruptor 4000) was used to uniformly disperse the nanoparticles in the solution. Next, 50 mg of GelCA was dissolved in the solution containing the curcumin-loaded polydopamine nanoparticles and was mixed with 100 μL of 0.6 wt % solution of the photoinitiator lithium phenyl-2,4,6-trimethylbenzoylphosphinate (LAP). Finally, the mixed solution was irradiated under 365 nm UV light at 25 mW cm-2 for 3 minutes, allowing GelCA to undergo photo-crosslinking reactions facilitated by the LAP and UV irradiation. GelCA underwent photo-crosslinking through the dimerization of alkene functional groups on cinnamic acid, forming the GelCA hydrogel. The preferred composition of the composite hydrogel prepared in the present invention is 5 wt % cinnamic acid-modified gelatin, 0.1 wt % curcumin-loaded polydopamine nanoparticles, 0.06 wt % photoinitiator LAP, dissolved in 1 mL of deionized water.

### Example 2

### [Mechanical Properties]

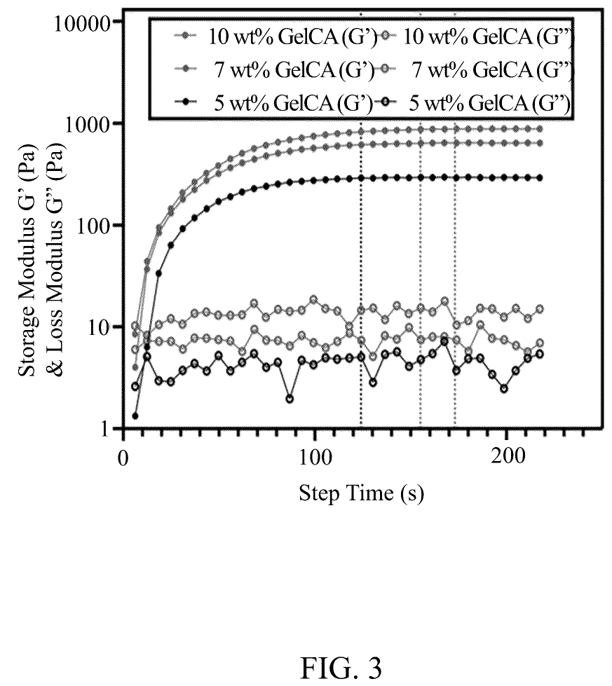

The mechanical properties of GelCA hydrogel formed through photo-crosslinking were investigated by selecting GelCA solutions at concentrations of 5, 7, and 10 wt % for rheological properties, compressive strength, and water absorption tests. As shown in FIG. 3, the rheological results were obtained for the 5, 7, and 10 wt % GelCA solutions under the influence of the photoinitiator and 25 mW cm-2 UV irradiation. The results demonstrated that all three concentrations of GelCA solutions exhibited a crossover between the storage modulus and the loss modulus during the initial stage of UV light exposure (within approximately 10 seconds), indicating that the transition of GelCA solution from a viscous material to a viscoelastic material under UV irradiation. Furthermore, with continued exposure to UV irradiation, the storage modulus of the 5 wt % GelCA solution reached a plateau (saturation curing point) at around 124 seconds, while the 7 and 10 wt % GelCA solutions at approximately 155 seconds and 173 seconds, respectively. The observations suggest that the GelCA in the solution reached the dimerization saturation curing point at these time points, forming structurally stable GelCA that could be used as a composite hydrogel matrix through photo-crosslinking.

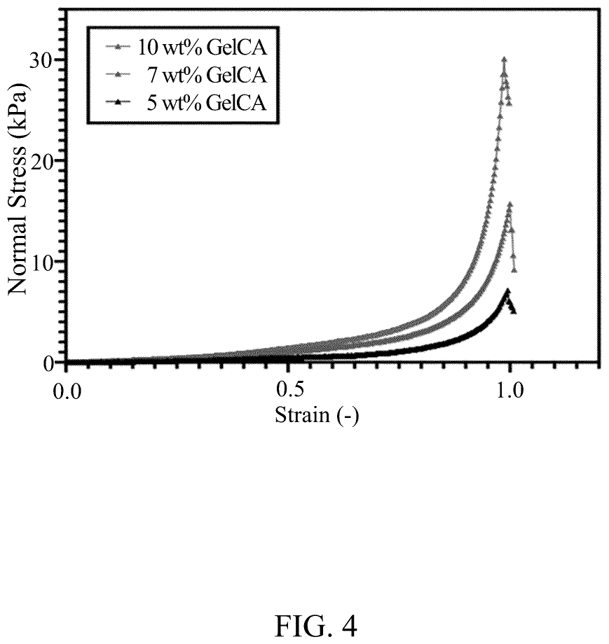

In addition, compressive strength was used to investigate the mechanical properties of GelCA at different weight percentages. As shown in FIG. 4, the stress-strain curves of GelCA hydrogels at three concentrations, after complete photopolymerization, demonstrated that all concentrations exhibited stable deformation under compressive stress, with structural failure occurring only when the strain reached approximately 99%, indicating that GelCA had excellent deformability. On the other hand, the Young's modulus of GelCA was measured at specific strain rates, with the Young's modulus of the 5 wt % hydrogels calculated to be 1.26±0.08 kPa, while the values for the 7 wt % and 10 wt % hydrogels were 3.26±0.06 kPa and 5.27±0.13 kPa, respectively. The results indicated that under the same stress, the lower concentration GelCA exhibited greater deformation, suggesting that the 5 wt % GelCA had better deformability.

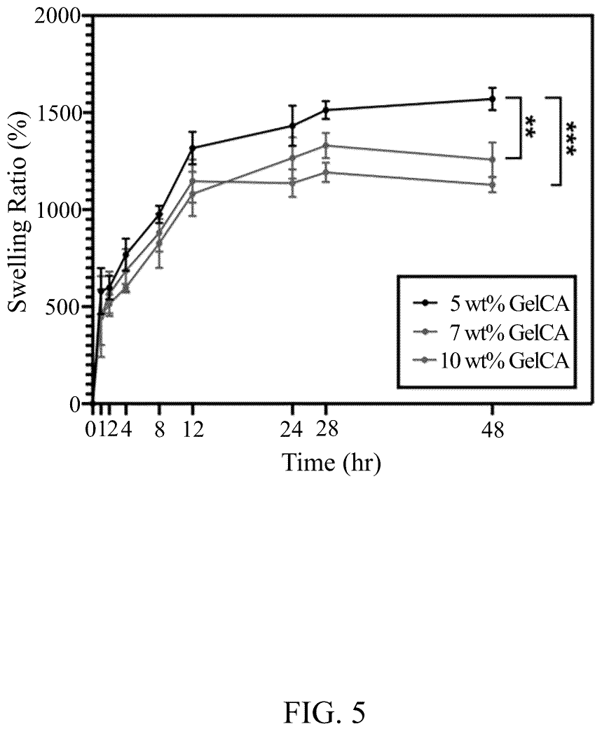

Photocrosslinked hydrogels had a 3D porous structure, which enabled them to absorb and retain a large amount of water. Therefore, swelling tests were conducted on GelCA at three different concentrations. FIG. 5 showed the liquid absorption ratios (swelling ratios) of GelCA at different concentrations. After rehydration for approximately 24 hours following lyophilization, the swelling ratios of all three concentrations of GelCA approached stability. After 48 hours, the swelling ratio of the 5 wt % GelCA reached the highest value among the three groups, approximately 1570%, indicating that this concentration of hydrogel had the highest liquid absorption capacity. This was likely due to a lower density of photo-crosslinking points, which resulted in lower hydrogel expansion constraint and higher porosity. Consequently, under similar osmotic pressure conditions, the 5 wt % GelCA exhibited the highest swelling ratio.

Based on the experimental results, it was found that the 5 wt % hydrogel matrix exhibited faster complete photocuring, greater compressive deformation, and a higher swelling ratio compared to other concentrations of GelCA. In subsequent in vivo experiments on the optic nerve crush (ONC) model, it was important that the hydrogel injected into the retina contains a higher percentage of water, making the hydrogel's water content closer to that of the natural vitreous tissue during delivery to the retinal tissue. Therefore, in the present invention, 5 wt % GelCA was selected for use in conjunction with polydopamine nanoparticles (PDA) and curcumin-loaded polydopamine nanoparticles (Cur@PDA).

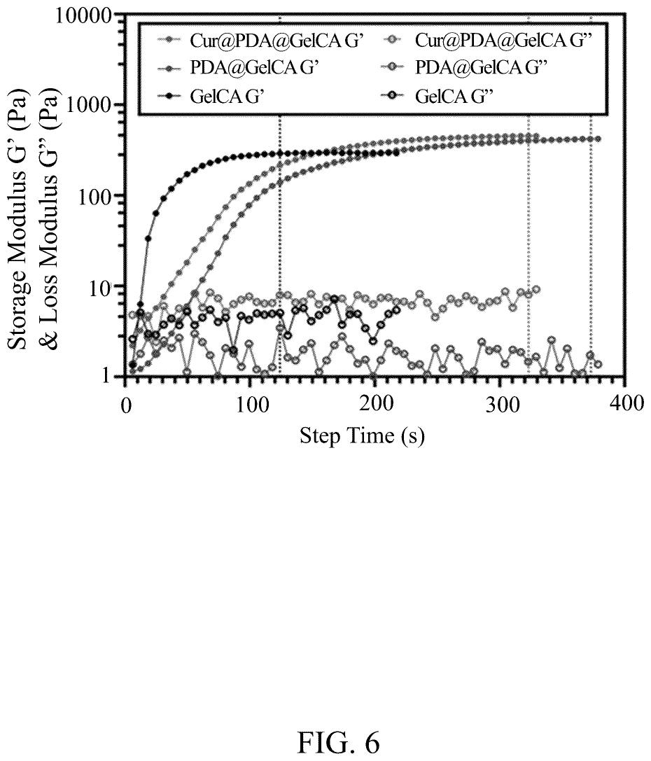

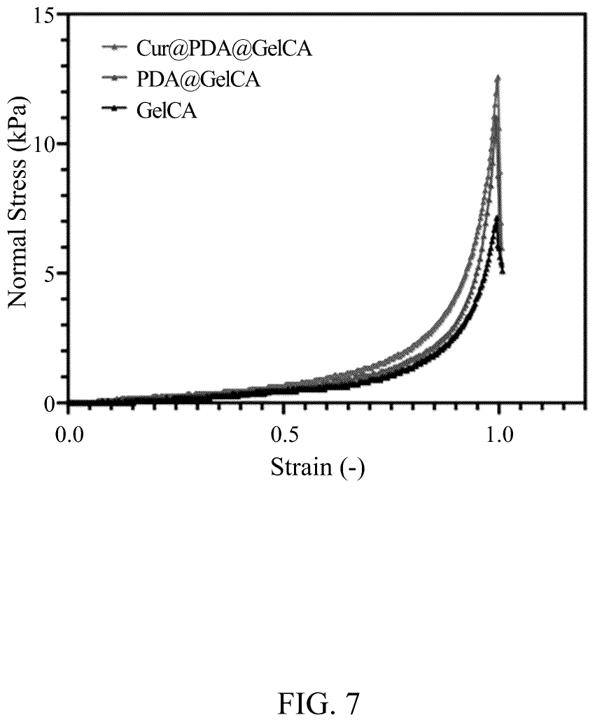

Further tests were conducted to assess the mechanical properties of GelCA hydrogels containing PDA and Cur@PDA nanoparticles (PDA@GelCA and Cur@PDA@GelCA). As shown in FIG. 6, after adding PDA and Cur@PDA nanoparticles to the 5 wt % GelCA nanocomposite, the crossover point between the loss modulus and storage modulus of PDA@GelCA was extended to approximately 31 seconds, while the crossover point of Cur@PDA@GelCA occurred at around 28 seconds. Additionally, the results showed that the incorporation of PDA and Cur@PDA nanoparticles increased the time required for the GelCA to reach complete photodimerization, with the time extending from 124 seconds in the GelCA group to 372 seconds in the PDA@GelCA group and 323 seconds in the Cur@PDA@GelCA group. This indicates that the presence of nanoparticles introduces spatial hindrance to GelCA dimerization. However, both PDA@GelCA and Cur@PDA@GelCA reached a stable storage modulus after a few minutes of UV irradiation, demonstrating that the presence of PDA and Cur@PDA nanoparticles did not affect the stability of the complete gelation process. Next, comparing the storage modulus of different groups after complete photodimerization, the final modulus of the GelCA group was approximately 291.1±1.9 Pa, while the moduli of the PDA@GelCA and Cur@PDA@GelCA groups were 418.8±0.8 Pa and 451.4±1.4 Pa, respectively. The results indicate that the rigidity of the GelCA hydrogel significantly increased after the addition of nanoparticles, with the Cur@PDA@GelCA group showing a greater enhancement effect. Furthermore, as shown in FIG. 7, the mechanical properties of the three hydrogels were evaluated through compressive strength tests. Under a strain of 15-20%, the Young's modulus of the PDA@GelCA group was 1.87±0.11 kPa, and that of the Cur@PDA@GelCA group was 2.09±0.08 kPa, both higher than the 1.26±0.08 kPa of the GelCA group. The compressive moduli of all three hydrogels were lower than those of real animal tissues (such as murine and porcine), indicating that the hydrogels in the present invention are softer than animal tissues, which suggests that when this hydrogel is injected into the retina, it is less likely to cause damage.

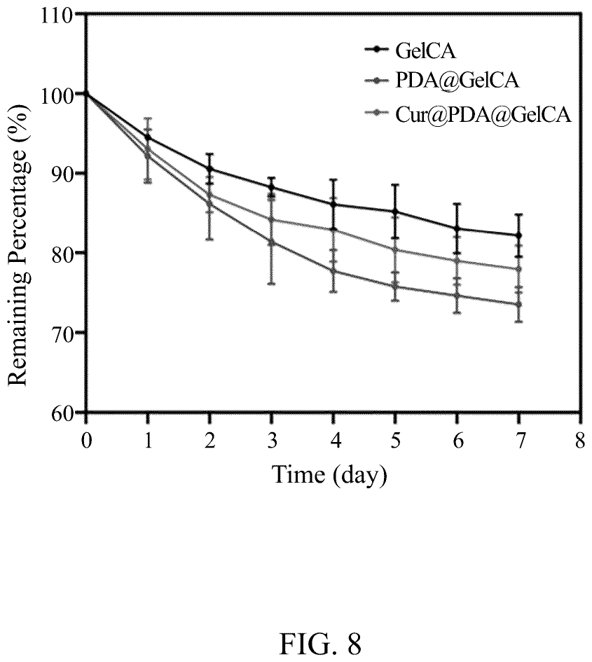

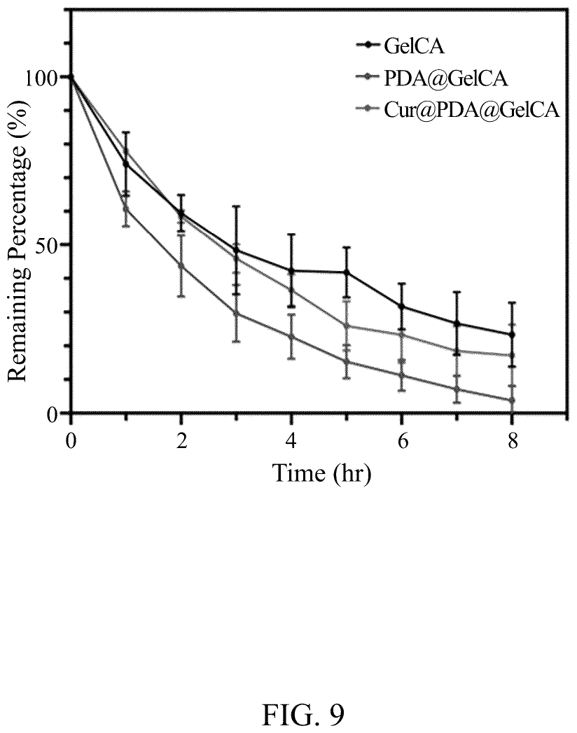

Furthermore, to determine the stability of the three hydrogels in a biological environment, long-term dissolution tests in a pure culture medium and short-term degradation tests with collagenase were conducted, as shown in FIG. 8 and FIG. 9. In the long-term dissolution test, the three hydrogels gradually dissolved in the culture medium over 7 days, releasing substances that were not covalently crosslinked within the GelCA structure, such as photoinitiators, uncrosslinked GelCA macromers, and nanoparticles. After the addition of nanoparticles, the time required for crosslinking to reach saturation increased, and more than 70% of the substances in the three hydrogels remained after 7 days in the culture medium. In the short-term degradation test, the collagenase type I enzyme was used to digest the structure of GelCA. The results showed that the presence of nanoparticles accelerated the hydrolysis rate of GelCA, but the GelCA and Cur@PDA@GelCA groups still retained about 20% of their structure. Therefore, the addition of nanoparticles reduced the available crosslinking sites in GelCA, particularly in the case of PDA nanoparticles, which accelerated the digestion of the hydrogel structure by the collagenase type I enzyme.

### Example 3

### [Reactive Oxygen Species (ROS) Scavenging Efficiency of Nanoparticles]

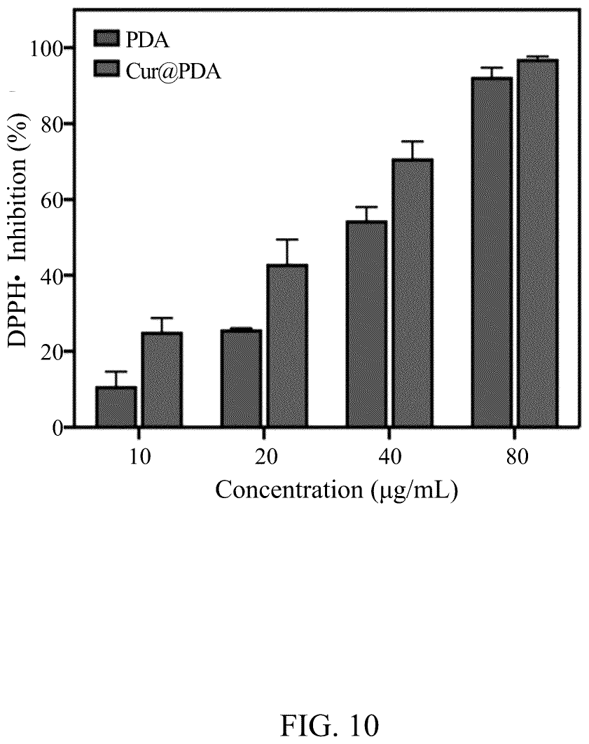

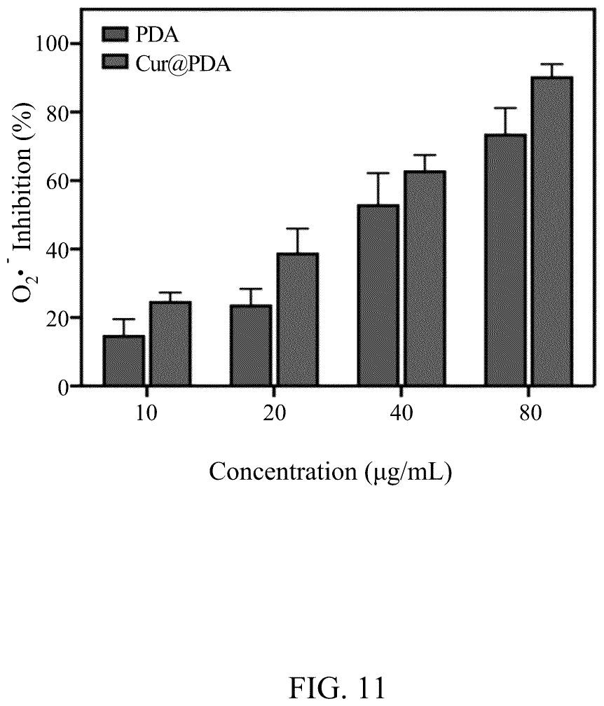

Please refer to FIG. 10 and FIG. 11. To verify the ROS scavenging efficiency of curcumin-loaded polydopamine nanoparticles, scavenging tests were conducted using free radicals (DPPH⋅) and superoxide radicals (O₂^{•—}). The results, as shown in FIG. 10, indicate that as the concentration increases, the inhibition effect of the polydopamine nanoparticles is enhanced, suggesting that the concentration of nanoparticles plays a significant role in free radical scavenging. At a high concentration (80 ug/mL), both groups nearly completely reduced the free radical DPPH⋅ in the solution. However, the curcumin-loaded polydopamine nanoparticles demonstrated significantly better DPPH⋅ scavenging efficiency at both low and high concentrations compared to the non-loaded polydopamine nanoparticles. Additionally, polydopamine nanoparticles also showed scavenging effects on other types of ROS. As shown in FIG. 11, both groups effectively reduced the concentration of O₂^{•—}, with the curcumin-loaded polydopamine nanoparticles removing 90% of O₂^{•—} at a high concentration (80 μg/mL), significantly outperforming the 70% removal by the non-loaded polydopamine nanoparticles. Similarly, the curcumin-loaded polydopamine nanoparticles exhibited superior O₂^{•—} scavenging efficiency at both low and high concentrations compared to the non-loaded polydopamine nanoparticles.

### Example 4

### [In Vitro Biocompatibility Test]

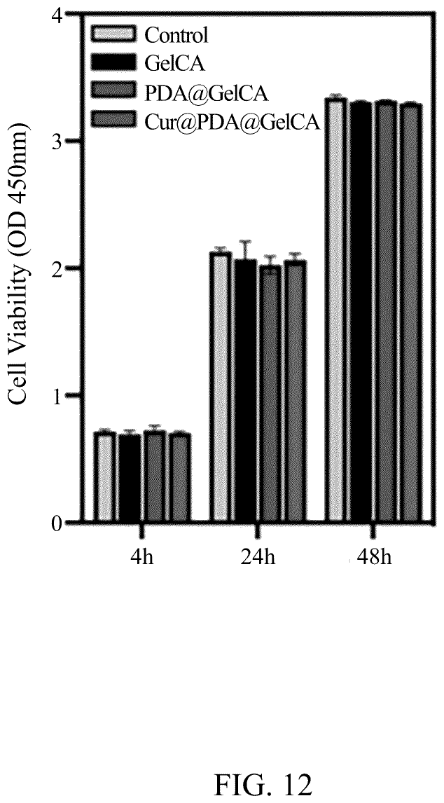

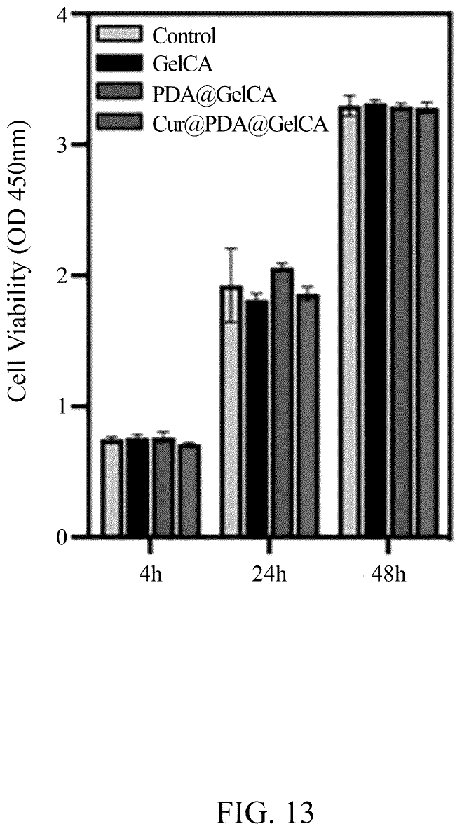

Next, to verify the in vitro biocompatibility of the three hydrogels, cell viability assays (CCK-8 assay) and live & dead fluorescence staining were conducted for quantitative and qualitative analysis of cell viability, evaluating the survival rates of mouse retinal ganglion cells RGC-5 and R661W cells. As shown in FIG. 12 and FIG. 13, the results of the CCK-8 assay indicated that after a certain period of cultivation, there was no significant difference in cell viability between the groups cultured with extracts from GelCA, PDA@GelCA, or Cur@PDA@GelCA hydrogels compared to the control group cultured with growth medium without any hydrogel. Furthermore, the live & dead cell staining results (not shown in the figures) of RGC-5 and R661W cells after cultivation in the presence of hydrogel extracts were consistent with the CCK-8 assay results. Therefore, it can be concluded that none of the three hydrogels exhibited significant cytotoxicity, meaning that the composite hydrogel of the present invention has good in vitro biocompatibility.

### [In Vivo Biocompatibility Test]

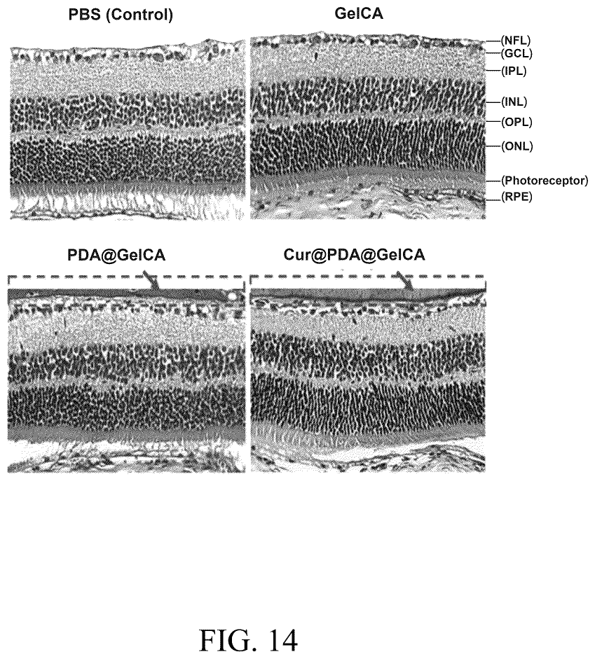

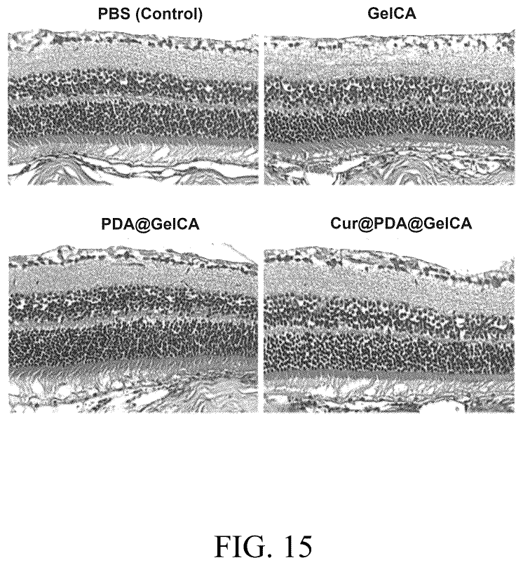

Please refer to FIG. 14 and FIG. 15. To evaluate the in vivo biocompatibility of the three different hydrogels, GelCA, PDA@GelCA, and Cur@PDA@GelCA, intravitreal injections were performed in healthy mice, and retinal samples were collected for evaluation 7 days and 14 days post-injection. FIG. 14 and FIG. 15 show the results of H&E staining after 7 days and 14 days, respectively. The results indicate that the PDA@GelCA and Cur@PDA@GelCA hydrogels exhibited excellent adhesion to the outer surface of the nerve fiber layer (NFL) (highlighted in red boxes) within the first 7 days post-injection, while the GelCA group did not exhibit adhesion on the NFL outer surface. This suggests that the addition of PDA and Cur@PDA nanoparticles enhances the adhesion of the hydrogels to the tissue surface, achieved through the strong T-T stacking and hydrogen bonding capabilities of the PDA nanoparticles. Additionally, compared to the PBS control group, the retinal tissues injected with the three hydrogels maintained the integrity of the major functional cell layers, such as the ganglion cell layer (GCL), inner nuclear layer (INL), and outer nuclear layer (ONL), and did not result in cell loss, tissue atrophy, or inflammatory responses at both 7 days and 14 days post-injection. Therefore, the GelCA, PDA@GelCA, and Cur@PDA@GelCA hydrogels exhibit excellent in vivo biocompatibility within retinal tissues, without causing tissue toxicity effects.

### [In Vitro Antioxidative Performance]

To further evaluate the in vitro antioxidant properties of PDA@GelCA and Cur@PDA@GelCA hydrogels, Amplex™ Red reagent (10-acetyl-3,7-dihydroxyphenoxazine) was used to assess oxidative stress in RGC-5 cells under the presence of hydrogen peroxide. Therefore, results from different concentrations of PDA and Cur@PDA nanoparticles (50, 100, and 200 μg) under the same hydrogen peroxide conditions were compared (results not shown). In the presence of GelCA, RGC-5 cells exhibited a resorufin signal similar to that of the H₂O₂ group. Additionally, the results from PDA@GelCA and Cur@PDA@GelCA showed that as the concentration of nanoparticles in the hydrogel increased, the resorufin signal in RGC-5 cells gradually weakened. These findings suggest that under certain concentrations of hydrogen peroxide, the in vitro antioxidant properties of PDA@GelCA and Cur@PDA@GelCA hydrogels can be distinguished.

### [In Vivo Antioxidative Performance]

Next, the in vivo antioxidant properties of PDA@GelCA and Cur@PDA@GelCA hydrogels were evaluated using the ONC (Optic Nerve Crush) model to simulate retinal oxidative stress in mice. The induction process of the ONC model triggers a series of cellular oxidative stress responses, potentially activating oxidases and leading to an increase in intracellular ROS production. Finally, the retinal tissues will undergo intravitreal injections of GelCA, PDA@GelCA, and Cur@PDA@GelCA to evaluate their antioxidant properties.

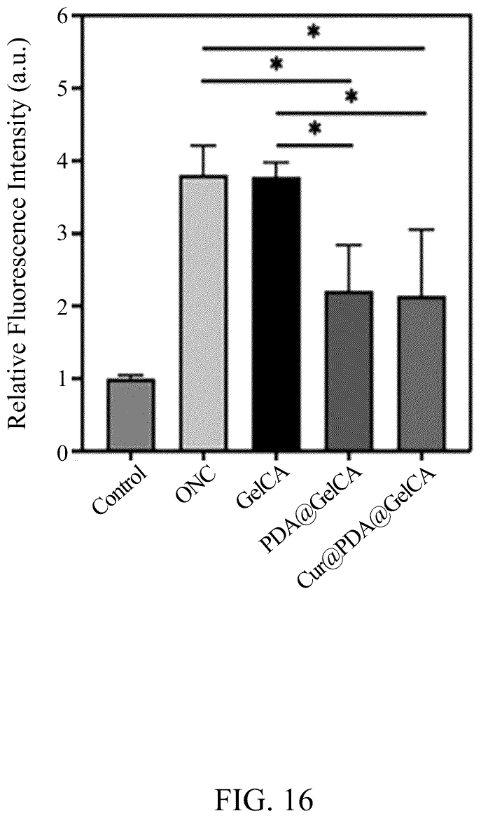

First, we used dihydroethidium (DHE) staining to evaluate the oxidative state of the retinal tissues. As shown in FIG. 16, the quantitative results of fluorescence intensity detected after DHE staining indicated that oxidative stress increased in the retinal tissues treated with ONC, with fluorescence intensity in the ONC group higher than that of the PBS control group. However, among the groups treated with the three types of hydrogels following ONC treatment, the fluorescence intensity in the GelCA hydrogel group was similar to that of the ONC group, showing no significant difference. In other words, GelCA alone had a limited impact on tissue antioxidant properties. In contrast, the groups treated with PDA@GelCA and Cur@PDA@GelCA hydrogels showed significant differences from the ONC group, suggesting that both hydrogels can further alleviate oxidative damage in tissues following ONC treatment.

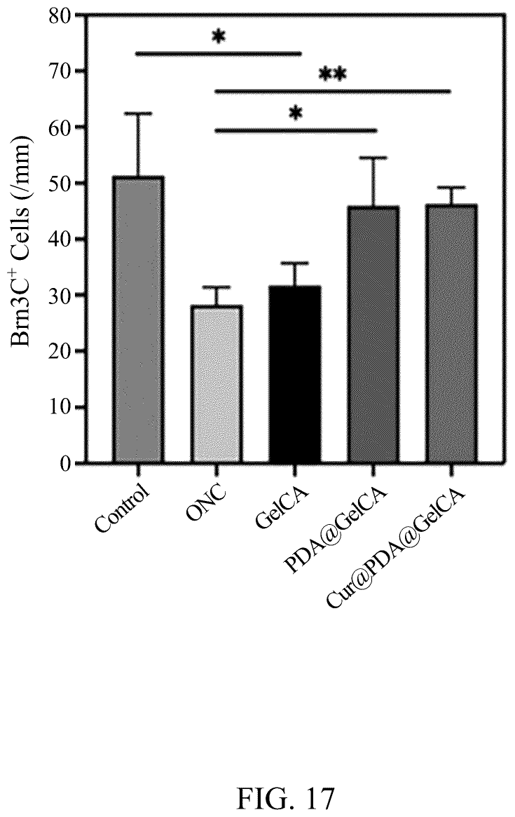

On the other hand, a Brn3C antibody, specific to retinal ganglion cells (RGCs), was used to label the ganglion cells in retinal tissues collected after in vivo injection. As shown in FIG. 17, quantitative results of the number of cells per millimeter in the GCL by immunofluorescence staining revealed that in the control group, which was only injected with PBS and did not undergo ONC treatment. The ONC group and the GelCA group, where PBS and GelCA hydrogel were injected respectively after ONC treatment, showed a significant reduction in Brn3C-positive cells, corresponding to a decrease in ganglion cells. In contrast, in the groups injected with PDA@GelCA and Cur@PDA@GelCA hydrogels following ONC treatment, the number of Brn3C-positive cells was close to that of the control group. This indicates that PDA@GelCA and Cur@PDA@GelCA hydrogels effectively mitigated the reduction in cell number, demonstrating that these hydrogels with PDA and Cur@PDA nanoparticles further inhibited excessive oxidative stress in retinal tissues following ONC treatment and prevented apoptosis in the GCL.

In summary, the composite hydrogel provided by the present invention comprises cinnamic acid-modified gelatin and polydopamine nanoparticles loaded with curcumin. The cinnamic acid-modified gelatin, upon treatment with a photoinitiator and subjected to photo-crosslinking, forms a photodimerized modified hydrogel that exhibits superior mechanical properties and acts as the hydrogel matrix in the composite hydrogel. Additionally, by incorporating curcumin, which is lipophilic and has low bioavailability, into the polydopamine nanoparticles, the bioavailability is increased, enhancing the ability to scavenge free radicals and superoxide radicals (ROS). Furthermore, the composite hydrogel of the present invention demonstrates excellent bioavailability and biocompatibility, as well as adhesion and prolonged degradation characteristics in tissues. This composite hydrogel can be effectively injected into retinal tissues, scavenging ROS and improving antioxidant properties, thereby contributing to the treatment of retinal oxidative damage.

Although the present invention has been described in considerable detail with reference to certain embodiments thereof, other embodiments are possible. Therefore, the spirit and scope of the appended claims should not be limited to the description of the embodiments contained herein.

What is claimed is:

1. A composite hydrogel, comprising:
a gelatin;
a cinnamic acid;
a plurality of polydopamine nanoparticles; and
a curcumin;
wherein the gelatin is chemically cross-linked with the cinnamic acid to form a cinnamic acid-modified gelatin, and the curcumin is linked by a non-covalent binding method to the polydopamine nanoparticles to form a plurality of curcumin-loaded polydopamine nanoparticles, the cinnamic acid-modified gelatin undergoes a photo-crosslinking reaction and then binds with the curcumin-loaded polydopamine nanoparticles to form the composite hydrogel.

2. The composite hydrogel as claimed in claim 1, wherein the gelatin is chemically cross-linked with the cinnamic acid by a chemical crosslinking agent forming an amide bond between the carboxyl group of the cinnamic acid and an amino group of the gelatin to form a covalent bond.

3. The composite hydrogel as claimed in claim 2, wherein the chemical crosslinking agent is 1-ethyl-3-(3-dimethylaminopropyl) carbodiimide (EDC).

4. The composite hydrogel as claimed in claim 1, further comprising a photoinitiator and a deionized water, and wherein the photoinitiator is lithium phenyl-2,4,6-trimethylbenzoylphosphinate (LAP).

5. The composite hydrogel as claimed in claim 4, wherein the photo-crosslinking reaction is initiated by an ultraviolet irradiation of the photoinitiator and generates a photo-dimerization reaction at the alkene functional group in the cinnamic acid-modified gelatin.

6. The composite hydrogel as claimed in claim 1, wherein the polydopamine nanoparticles are formed through the self-polymerization of a dopamine.

7. The composite hydrogel as claimed in claim 1, wherein based on the total weight of the composite hydrogel, the weight percentage of the curcumin-loaded polydopamine nanoparticles is less than 10 wt %.

8. The composite hydrogel as claimed in claim 1, wherein the non-covalent binding method includes-x stacking and hydrogen bonding.

9. The composite hydrogel as claimed in claim 1, wherein the composite hydrogel is injectable.

10. A method for treating retinal tissue damage, comprising administering to a subject in need thereof an effective amount of the composite hydrogel of claim 1.
